(19) United States
(12) Patent Application Publication
(10) Pub. No.: US 2026/0020947 A1
(43) Pub. Date: Jan. 22, 2026

(54) FIXATION OF DEBRIS PROTECTION DEVICES IN BODY LUMENS

(71) Applicant: Zeev BRANDEIS, Rosh HaAyin (IL)

(72) Inventor: Zeev BRANDEIS, Rosh HaAyin (IL)

(21) Appl. No.: 19/106,246

(22) PCT Filed: Aug. 24, 2023
(86) PCT No.: PCT/IL2023/050900

§ 371 Date: Feb. 25, 2025

Related U.S. Application Data
(60) Provisional application No. 63/400,779, filed on Aug. 25, 2022.

(51) Int. Cl. A61F 2/01

(52) U.S. Cl. CPC A61F 2/0105; A61F 2002/016; A61F 2220/0008

(57) ABSTRACT

A device for anchoring a medical tool in a large body lumen, the device including an elongate support configured to axially support a medical tool in a body lumen against longitudinal movement along the body lumen, wherein the elongate support is axially inflexible and can bend laterally. An anchoring frame for anchoring in a body lumen, the frame sized and shaped to expand to a diameter corresponding to the aorta. Related apparatus and methods are also described.

### RELATED APPLICATIONS

This application is a PCT application which claims the benefit of priority of U.S. Provisional Patent Application No. 63/400,779 filed 25 Aug. 2022 titled “Fixation of Debris Protection Devices in Body Lumens” and is related to U.S. Provisional Patent Application No. 63/398,546 filed on Aug. 17, 2022, titled “CAPTURING FLOWING DEBRIS IN BLOOD VESSELS AND OTHER BODY LUMENS”.

This application is also related to U.S. Provisional Patent Application No. 63/400,771 filed 25 Aug. 2022, titled “PROTECTING CORONARY ARTERIES AND MYOCARDIUM BLOOD VESSELS DURING MEDICAL PROCEDURES”, U.S. Provisional Patent Application No. 63/400,773 filed 25 Aug. 2022, titled “MANAGING DEBRIS-PROTECTION IN RELATION TO VASCULAR MEDICAL PROCEDURES OR MEDICAL PROCEDURES TO A HEART” and U.S. Provisional Patent Application No. 63/400,866 filed 25 Aug. 2023, titled “CAPTURING FLOWING DEBRIS IN THE VENA CAVA”.

The contents of all of the above applications are incorporated by reference as if fully set forth herein.

### FIELD AND BACKGROUND

The present disclosure, in some embodiments thereof, relates to mechanisms and methods for fixing and/or anchoring tubular devices in body lumens, and, more particularly, but not exclusively, to mechanisms and methods for fixing and/or anchoring debris protection devices in body lumens, and, even more particularly, but not exclusively, to mechanisms and methods for fixing and/or anchoring debris protection devices in an aorta.

### Additional Background Art Includes

International Patent Application Publication Number WO 2019/064223 of Brandeis, which describes an aortic protection device including a mesh lumen shaped and sized to extend along the aorta, from a heart-side of a brachiocephalic artery exit from the aorta to distal of a left subclavian artery exit from the aorta, wherein the mesh lumen is arranged to change a porosity of mesh pores in response to external control.

International Patent Application Publication Number WO 2017/042808 of Eli, which describes an embolic protection device including a porous deflector screen including a filter, arranged to expand and to conform to a wall of the aortic arch covering entrances to arteries branching from an aorta, an emboli collector including a cylinder arranged to expand and to lie along walls of a descending aorta, pushing against walls of the descending aorta and anchoring the porous deflector screen, and a connecting portion for connecting the porous deflector screen and the emboli collector, arranged to push the porous deflector screen against a wall of the aortic arch while anchoring against the emboli collector.

The disclosures of all references mentioned above and throughout the present specification, as well as the disclosures of all references mentioned in those references, are hereby incorporated herein by reference.

### SUMMARY

The present disclosure, in some embodiments thereof, relates to mechanisms and methods for fixing and/or anchoring tubular devices in body lumens, and, more particularly, but not exclusively, to mechanisms and methods for fixing and/or anchoring debris protection devices in body lumens, and, even more particularly, but not exclusively, to mechanisms and methods for fixing and/or anchoring debris protection devices in an aorta.

According to an aspect of some embodiments of the present disclosure there is provided a device for anchoring a medical tool in a large body lumen, the device including an elongate support configured to axially support a medical tool in a body lumen against longitudinal movement along the body lumen, wherein the elongate support is axially inflexible and can bend laterally.

According to some embodiments of the disclosure, the large body lumen is an aorta. According to some embodiments of the disclosure, the device includes a plurality of supports.

According to some embodiments of the disclosure, the support includes a hollow tube.

According to some embodiments of the disclosure, the support includes a hollow tube and the hollow tube contains a control wire for controlling the medical tool.

According to some embodiments of the disclosure, the medical tool includes a blood filtration mesh.

According to some embodiments of the disclosure, the medical tool includes a device for trapping debris flowing along the body lumen.

According to some embodiments of the disclosure, the medical tool includes a device for blocking debris from entering coronary arteries.

According to some embodiments of the disclosure, the medical tool includes a device for blocking debris from entering arteries leading to a brain.

According to some embodiments of the disclosure, the medical tool includes a device for trapping debris located in a vein.

According to some embodiments of the disclosure, the medical tool includes a device for trapping debris located in a Vena Cava.

According to an aspect of some embodiments of the present disclosure there is provided a system including a filter and an anchor, wherein the anchor includes a hollow tube is axially inflexible and can bend laterally.

According to some embodiments of the disclosure, the tube is connected to the filter at or near a distal end of the filter.

According to some embodiments of the disclosure, the tube is connected to the filter at or near a distal end of the tube.

According to an aspect of some embodiments of the present disclosure there is provided a method for anchoring a medical tool in a body lumen, the method including pushing a first medical tool supported by a support into a body lumen, wherein the support is longitudinally inflexible and laterally flexible, preventing longitudinal movement of the first medical tool along the body lumen.

According to some embodiments of the disclosure, the first medical tool includes a filter.

According to some embodiments of the disclosure, the support is attached to the first medical tool.

According to some embodiments of the disclosure, the support includes a hollow tube. According to some embodiments of the disclosure, the hollow tube contains a control wire for controlling the first medical tool.

According to some embodiments of the disclosure, the hollow tube contains a control wire for controlling a second medical tool.

According to some embodiments of the disclosure, the medical tool includes a blood filtration mesh.

According to some embodiments of the disclosure, the medical tool includes a device for trapping debris flowing along the body lumen.

According to some embodiments of the disclosure, the medical tool includes a device for blocking debris from entering coronary arteries.

According to some embodiments of the disclosure, the medical tool includes a device for blocking debris from entering arteries leading to a brain.

According to some embodiments of the disclosure, the medical tool includes a device for trapping debris located in a vein.

According to some embodiments of the disclosure, the medical tool includes a device for trapping debris located in a Vena Cava.

According to an aspect of some embodiments of the present disclosure there is provided an anchoring frame for anchoring in a body lumen, the frame sized and shaped to expand to a diameter corresponding to the aorta.

According to some embodiments of the disclosure, the anchoring frame sized and shaped to expand to a diameter corresponding to an adult aorta.

According to some embodiments of the disclosure, the anchoring frame sized and shaped to expand to a diameter corresponding to the ascending aorta between coronary arteries and a brachiocephalic artery.

According to some embodiments of the disclosure, the anchoring frame sized and shaped to expand to a diameter corresponding to the Vena Cava.

According to some embodiments of the disclosure, including two concentric layers.

According to some embodiments of the disclosure, configured so that a first, external layer is extruded first from a catheter, and a second internal layer is extruded second from the catheter and presses outward on the external layer.

According to some embodiments of the disclosure, including a longitudinally stiff wire configured to connect to a debris protection device placed upstream of the anchoring frame.

According to some embodiments of the disclosure, connected to a debris protection device placed upstream of the anchoring frame.

According to some embodiments of the disclosure, including a wire configured to connect to a debris protection device placed downstream of the anchoring frame.

According to some embodiments of the disclosure, connected to a debris protection device placed downstream of the anchoring frame.

According to some embodiments of the disclosure, configured so that when the frame is expanded, the frame leaves room for a medical tool to pass along a middle of the frame.

According to some embodiments of the disclosure, configured to expand under radial pressure and exert force against walls of the body lumen both radially and longitudinally along the body lumen.

According to some embodiments of the disclosure, the frame includes a locking mechanism which locks the frame in an expanded state.

According to some embodiments of the disclosure, the frame includes a protective membrane on an outer surface of the anchoring frame.

According to some embodiments of the disclosure, an outside wall of the frame is covered with a material for reducing damage to blood vessel walls.

According to some embodiments of the disclosure, an outside wall of the frame is covered with polymer.

According to some embodiments of the disclosure, an inside wall of the frame is covered with polymer.

According to some embodiments of the disclosure, configured to be retrieved from a patient's body without releasing debris from walls of the lumen.

According to some embodiments of the disclosure, configured to be retrieved from a patient's body without damaging walls of the lumen.

According to some embodiments of the disclosure, configured to have a star shaped cross section which expands to a tubular cross section to exert radially-outward force on walls of the body lumen.

According to some embodiments of the disclosure, including a mechanism internal to outer walls of the frame having a star shaped cross section which expands to a tubular cross section to exert radially-outward force on the frame.

According to some embodiments of the disclosure, including a mechanism internal to outer walls of the frame configured to exert radially-outward force on the frame.

According to some embodiments of the disclosure, including a hexagonally shaped mechanism internal to outer walls of the frame configured to exert radially-outward force on the frame.

According to some embodiments of the disclosure, further including a wire attached at one end to the anchoring frame, and at another end to a blood filtration mesh, so as to prevent the mesh from being swept downstream when the frame is fully expanded.

According to some embodiments of the disclosure, further including a wire attached at one end to the anchoring frame, and at another end to a device for trapping debris in an aorta, so as to prevent the device from being swept downstream when the frame is fully expanded.

According to some embodiments of the disclosure, the anchoring frame, when expanded against walls of an ascending aorta, is configured to withstand a force of 10-30 Newton pulling on the anchoring frame in a direction parallel to surface of the walls of the ascending aorta without being shifted along the walls.

According to some embodiments of the disclosure, including a control mechanism for controlling radial force exerted on walls of the body lumen from outside a body of a patient.

According to an aspect of some embodiments of the present disclosure there is provided a method for anchoring a medical tool in a body lumen, the method including inserting a first medical tool attached to an anchoring frame into a body lumen, locating the first medical tool at a desired location along the body lumen, expanding the frame to contact walls of the body lumen and exert an anchoring force on the walls of the body lumen.

According to some embodiments of the disclosure, the anchoring frame is attached to a wire for supporting the first medical tool upstream of the anchoring frame, and the expanding the frame includes expanding the frame downstream of the medical tool, such that the anchoring frame supports the first medical tool from shifting from the desired location.

According to some embodiments of the disclosure, the anchoring frame is attached to a wire for supporting the first medical tool downstream of the anchoring frame, and the expanding the frame includes expanding the frame upstream of the first medical tool, such that the anchoring frame supports the medical tool from shifting from the desired location.

According to some embodiments of the disclosure, further including passing a second medical tool to pass along a middle of the frame.

According to some embodiments of the disclosure, expanding the frame includes activating a locking mechanism which locks the frame in an expanded state.

According to some embodiments of the disclosure, further including removing the anchoring frame from a patient's body.

According to some embodiments of the disclosure, removing the anchoring frame from a patient's body includes removing without releasing debris from walls of the lumen.

According to some embodiments of the disclosure, removing the anchoring frame from a patient's body includes removing without damaging walls of the lumen.

According to some embodiments of the disclosure, the expanding the frame to contact walls of the body lumen and exert an anchoring force on the walls of the body lumen includes expanding to exert a force sufficient to withstand a torque in a range of 10-30 Newton meter pulling on the anchoring frame in a direction parallel to surface of the walls of body lumen without being shifted along the walls.

According to some embodiments of the disclosure, the expanding the frame to exert a force sufficient to withstand a torque in a range of 10-30 Newton meter includes operating a control mechanism to exert the force.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the disclosure are herein described, by way of example only, with reference to the accompanying drawings and images. With specific reference now to the drawings and images in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

In the drawings:

FIG. 1A is a simplified line drawing illustration of a debris protection device such as described in above-mentioned International Patent Application Publication Number WO 2019/064223 of Brandeis, which potentially benefits from anchoring according to an example embodiment;

FIG. 1B is a simplified line drawing illustration of a debris protection device such as described in above-mentioned co-filed U.S. Provisional Patent Application titled “Protecting Coronary Arteries and Myocardium Blood Vessels During Medical Procedures” (Attorney Docket Number 91186), which potentially benefits from anchoring according to an example embodiment;

FIG. 1C is a simplified line drawing illustration of a debris protection device such as described in above-mentioned co-filed U.S. Provisional Patent Application titled “Protecting Coronary Arteries and Myocardium Blood Vessels During Medical Procedures” (Attorney Docket Number 91186), which potentially benefits from anchoring according to an example embodiment;

FIG. 1D is a simplified line drawing illustration of a debris protection device such as described in above-mentioned U.S. Provisional Patent Application No. 63/398,546, which potentially benefits from anchoring according to an example embodiment;

FIG. 1E is a picture of a device which optionally includes aortic protection and debris trapping such as described in above-mentioned U.S. Provisional Patent Application No. 63/398,546 which potentially benefits from anchoring according to an example embodiment;

FIG. 1F is a simplified line drawing illustration of a device which optionally includes debris trapping such as described in above-mentioned U.S. Provisional Patent Application No. 63/398,546 which potentially benefits from anchoring according to an example embodiment;

FIGS. 2A-2D are photographs of a mechanism for anchoring according to an example embodiment;

FIGS. 2E-2F are photographs of a mechanism for anchoring according to an example embodiment;

FIG. 3A is a simplified illustration of a support and a control wire passing therethrough according to an example embodiment;

FIG. 3B is a simplified illustration of an anchoring frame and supports and/or control wires according to an example embodiment;

FIG. 4A is a simplified illustration of an anchoring frame exiting a catheter according to an example embodiment;

FIG. 4B is a simplified illustration of an anchoring frame according to an example embodiment;

FIG. 4C is a simplified illustration of an anchoring frame according to an example embodiment;

FIG. 4D is a simplified illustration of an anchoring frame according to an example embodiment;

FIG. 5 is a simplified flow chart illustration of a method for anchoring a medical tool in a body lumen according to an example embodiment;

FIGS. 6A-6G are drawings of an anchoring frame according to an example embodiment;

FIGS. 7A-7F are drawings of an inner layer of an anchoring frame according to an example embodiment;

FIG. 8 is a simplified flow chart illustration of a method of fixation including controlling an addition of additional, extra, radial force according to an example embodiment;

FIG. 9 is a simplified flow chart illustration of a method of fixation including self-expansion and controlling an addition of additional, extra, radial force according to an example embodiment; and

FIG. 10 is a simplified line drawing illustration of a debris protection device which potentially benefits from anchoring according to an example embodiment.

### DESCRIPTION OF SPECIFIC EMBODIMENTS

The present disclosure, in some embodiments thereof, relates to mechanisms and methods for fixing and/or anchoring tubular devices in body lumens, and, more particularly, but not exclusively, to mechanisms and methods for fixing and/or anchoring debris protection devices in body lumens, and, even more particularly, but not exclusively, to mechanisms and methods for fixing and/or anchoring debris protection devices in an aorta.

Before explaining at least one embodiment of the disclosure in detail, it is to be understood that the disclosure is not necessarily limited in its application to the details of construction and the arrangement of the components and/or methods set forth in the following description and/or illustrated in the drawings and/or the Examples. The disclosure is capable of other embodiments or of being practiced or carried out in various ways.

### Overview

Various medical procedures or devices are inserted into body lumens, and need to be placed there and stay in place.

Some such devices are inserted to stay in place for good, or at least for a long time. For example support stents.

Some such devices are inserted to stay in place for a limited time—by way of a non-limiting example inserting one or more debris-protection devices such as: an aortic protection device such as described in above-mentioned International Patent Application Publication Number WO 2019/064223 of Brandeis, and/or a debris capture device such as described in above-mentioned U.S. Provisional Patent Application No. 63/398,546, and/or in above mentioned co-filed U.S. Provisional Patent Application titled “Protecting Coronary Arteries and Myocardium Blood Vessels During Medical Procedures” (Attorney Docket Number 91186).

Various embodiments described herein relate to long term fixation or anchoring of medical devices in a body lumen, and various embodiments described herein relate to temporary fixation or anchoring of medical devices in a body lumen

It is desirable to secure structural elements—stents, stent grafts and endovascular repair elements, either permanent devices or temporary devices, that take part in medical procedures and more particularly, but not exclusively, to devices and methods to protect such devices from movement, migration or propagation during medical procedures.

Stents and stent grafts that are used to repair aortic endovascular injury\disease such as aortic aneurism, aortic dissection, aortic coarctation, dilated aorta, aortic leakage\rupture—are known to migrate, move and change their position in a deployed area. Various solutions are hereby described to fixate the stent\stent graft to its deployed position. These solutions take into consideration the vulnerability of the aorta (Shaggy aorta, porcelain aorta).

It is noted that fixation devices and methods as described herein are also used as add-ons to existing stent grafts, either as temporary add-ons or as permanent add-ons.

In some embodiments, fixation devices and methods as described herein are configured to allow other medical tools i.e. catheters, guide wires, delivery systems, and drug delivery systems to pass along or through the fixation.

### Devices

An aspect of some embodiments relates to devices and methods for anchoring a medical device in a body lumen by expansion of a frame, to exert force radially outward on walls of the body lumen.

In some embodiments, the force is optionally spread equally over the area of contact of the device to the body lumen wall. By way of a non-limiting example, taking a cylindrical area of contact, the force is optionally spread equally over the area of the cylinder—the area being 2πR×length, where R is a radius of the cylinder and length is the length of the cylinder.

In some embodiments, a shape of the frame is optionally configured to conform to an inside shape of the body lumen—for example to have a non-round cross section, and/or to conform longitudinally to a curved body lumen, such as the aortic arch. In some embodiments, an outer surface of the frame optionally follows actual topography of an inner surface of the body lumen.

In some embodiments, the frame is configured to expand at least to a diameter of a rather large body lumen, for example an aorta, even an adult aorta, or a Vena Cava.

In some embodiments, the frame is configured to exert pressure onto walls of a body lumen in two steps. A first step exerts some radial pressure, and assists in keeping a location of the frame in the body lumen. A second step exerts additional radial pressure, enabling the frame to provide support against shifting downstream by pressure of flowing fluid.

In some embodiments, the frame is fixated in a body lumen against shifting downstream.

In some embodiments, the frame include an attachment, and/or an attachment wire, to connect to some medical device and fixate the medical device against shifting downstream.

By deploying the fixation in two steps, a potential impact to the walls of the body lumen\aorta\tubular vessel is optionally reduced, preventing potential leakage, rupture, scratching, detachment of particles—plaque, calcifications, organized blood clots, injuring aorta sections that are rigid and may be fragile.

An aspect of some embodiments relates to devices and methods for anchoring a medical device in a body lumen by supporting the device longitudinally by supports configured not to flex in a longitudinal direction, even if they do flex side-to-side, to pass through various body lumens on a path to a desired location.

The support or support are configured to be longitudinally stiff, to prevent a supported device from being gradually shifted downstream by pressure of flowing fluid such as blood. In some embodiments, the support or supports are configured to be able to bend laterally, so as to be maneuvered into body lumens and along their twists and turns.

In some embodiments, the longitudinal support may actually be more than one longitudinal support, but rather 2, 3, 4, 5, 6, 7, 8 and so on, whole number up to, for example, 25.

In some embodiments, one or more of the longitudinal supports may optionally be shaped as tubes, through which control wires may optionally be passed, and used to control medical devices.

In some embodiments, the tube-shaped longitudinal support(s) may optionally be shaped as coiled springs, which provide longitudinal support and can flex laterally.

In some embodiments, the frame may optionally be similar to a stent-like device.

Various materials are contemplate for use in fixation device as described herein, including polymer struts, polymer poles, extruded poles\struts, pultrusion poles\struts, fiber reinforced poles\struts, wires, a combination of metal and polymer fibers\struts poles, use of biodegradable parts for temporary extra fixation, use of bio-modified tissue in addition to fixation.

### Location of Anchoring/Fixation

An example location for anchoring and/or fixation of the anchoring/fixation device, when used for anchoring in the ascending aorta, may be upstream of the innominate artery.

An example location for anchoring and/or fixation of the anchoring/fixation device, when used for anchoring in the ascending aorta, may be upstream of the innominate artery and downstream of the coronary arteries.

### Timing and/or Duration of Anchoring/Fixation

As mentioned above, various anchoring/fixation device embodiments are inserted into body lumens to stay in place for a long duration—a duration of days, weeks, months, years after the insertion.

A timing of inserting such anchoring/fixation devices may optionally be prior to performing a medical procedure in which it is desired to anchor debris-protection devices such as mentioned above, and/or following a medical event which is known or expected to produce blood-borne debris.

As mentioned above, various anchoring/fixation device embodiments are inserted into body lumens to stay in place temporarily—for example for a duration of a medical procedure known or expected to produce blood-borne debris.

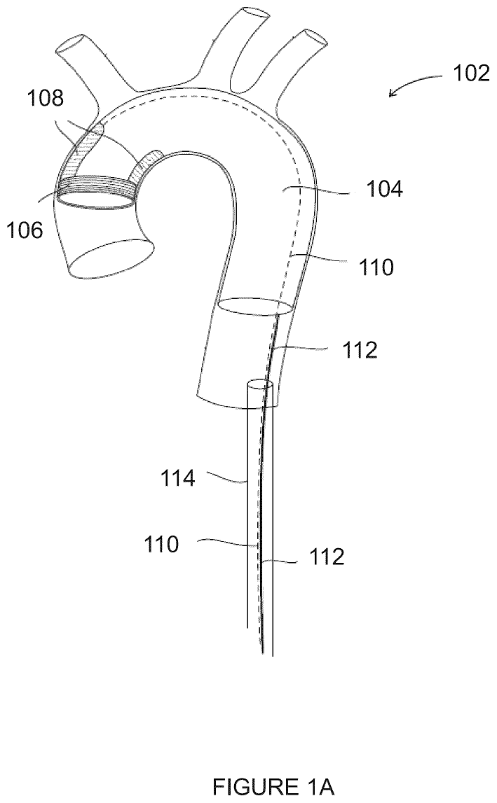

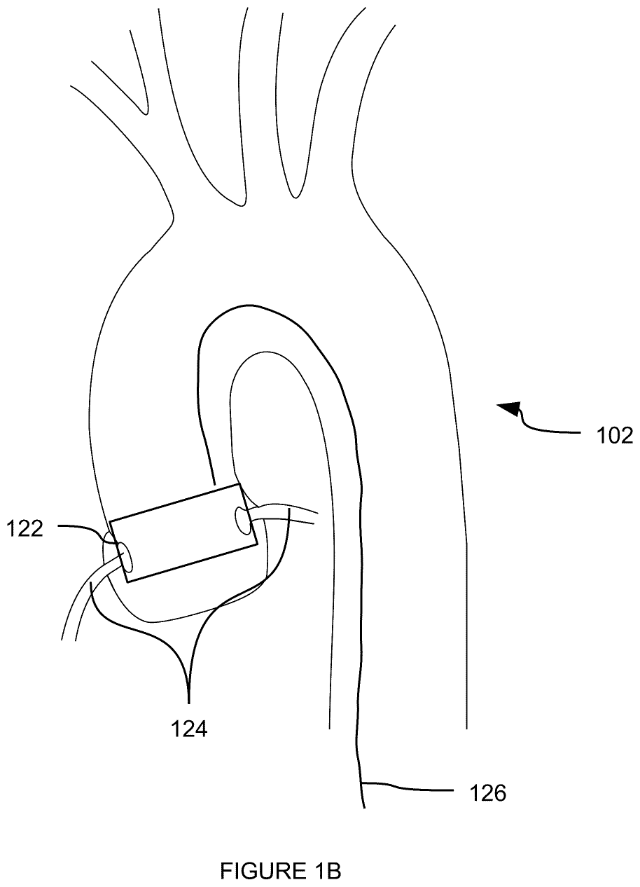

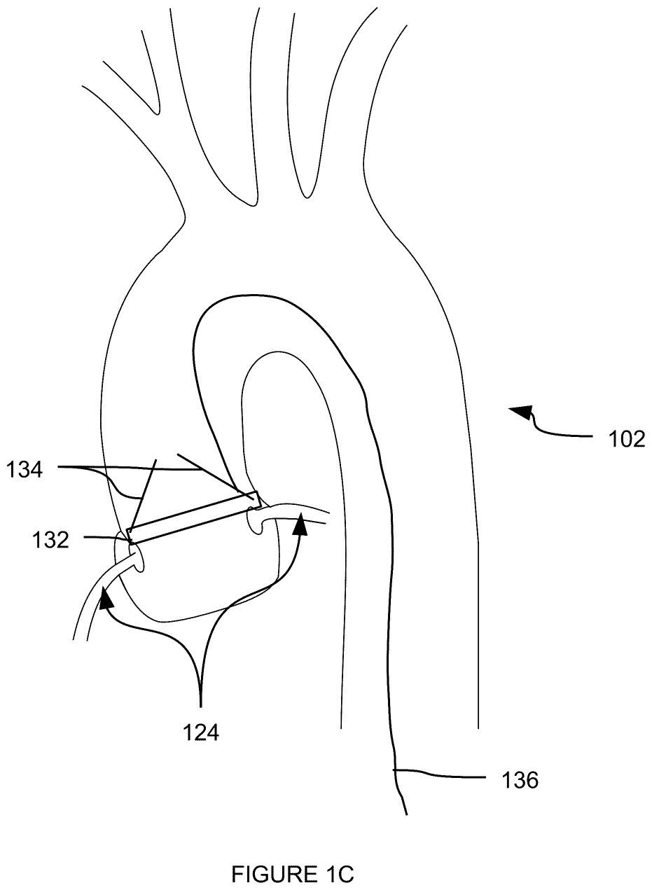

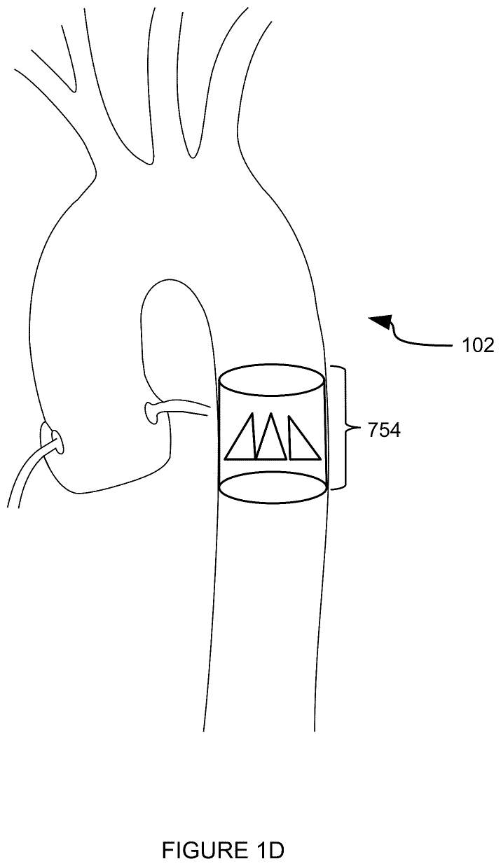

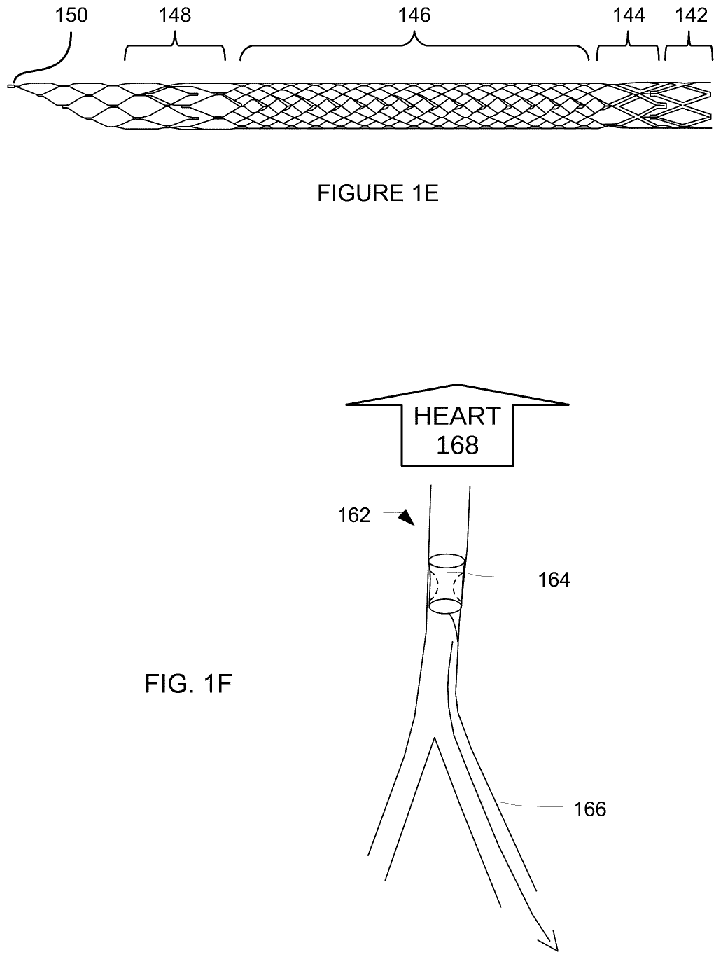

Reference is now made to FIG. 1A, which is a simplified line drawing illustration of a debris protection device such as described in above-mentioned International Patent Application Publication Number WO 2019/064223 of Brandeis, which potentially benefits from anchoring according to an example embodiment.

FIG. 1A shows an aortic protection device deployed in an aorta 102, the aortic protection device having two mesh layers—a first mesh layer 104 and a compactly packed second mesh layer 108.

FIG. 1A also shows an expandable anchoring frame 106 at a heart-proximal side of the aortic protection device, a first control wire 112 attached to a heart-distal end of the device, and a second control wire 110 attached to a heart-proximal end of the device and/or to the expandable anchoring frame 106, and a tube 114 through which the first control wire 112 and the second control wire 110 pass to outside a body of a patient.

Reference is now made to FIG. 1B, which is a simplified line drawing illustration of a debris protection device such as described in above-mentioned co-filed U.S. Provisional Patent Application titled “Protecting Coronary Arteries and Myocardium Blood Vessels During Medical Procedures” (Attorney Docket Number 91186), which potentially benefits from anchoring according to an example embodiment.

FIG. 1B shows a simplified schematic block illustration of a coronary artery 124 protection device 122 deployed in an aorta 102, the coronary artery protection device connected to a control wire 126.

In some embodiments, the coronary artery protection device 122 includes anchoring by an expandable frame as described herein.

Reference is now made to FIG. 1C, which is a simplified line drawing illustration of a debris protection device such as described in above-mentioned co-filed U.S. Provisional Patent Application titled “Protecting Coronary Arteries and Myocardium Blood Vessels During Medical Procedures” (Attorney Docket Number 91186), which potentially benefits from anchoring according to an example embodiment.

FIG. 1C shows a simplified schematic block illustration of a coronary artery 124 protection device 132 deployed in an aorta 102, the coronary artery protection device 124 including a one way valve 134, and the coronary artery protection device connected to a control wire 136.

In some embodiments, the coronary artery protection device 132 includes anchoring by an expandable frame as described herein.

Reference is now made to FIG. 1D, which is a simplified line drawing illustration of a debris protection device such as described in above-mentioned U.S. Provisional Patent Application No. 63/398,546, which potentially benefits from anchoring according to an example embodiment.

FIG. 1D shows a simplified schematic block illustration of a debris trapping device 754 deployed in an aorta 102.

In some embodiments, the debris trapping device 754 includes anchoring by an expandable frame as described herein.

In some embodiments, the debris trapping device 754 includes longitudinal support as described herein.

Reference is now made to FIG. 1E, which is a picture of a device which optionally includes aortic protection and debris trapping such as described in above-mentioned U.S. Provisional Patent Application No. 63/398,546 which potentially benefits from anchoring according to an example embodiment.

FIG. 1E shows a device which includes an anchoring or fixation section 142, a first debris-trapping section 144, a frame lumen 146, a second debris-trapping section 148, and an optional gradually-narrowing end 150 of the device.

In some embodiments, the device optionally includes a mesh (not shown) covering the frame lumen 146. The mesh optionally acts to filter debris so as not to enter branching arteries, for example as described in above-mentioned International Patent Application Publication Number WO 2019/064223 of Brandeis.

For the sake of clarity of the picture, FIG. 1E does not show an optional control wire or wires which may be used to control one or more of:
activating and deactivation the anchoring or fixation action of the anchoring or fixation section 142;
activating and deactivation the debris trapping of the first debris-trapping section 144;
changing mesh size of an optional mesh covering the frame lumen 146;
activating and deactivation the debris trapping of the second debris-trapping section 148; and
pushing or pulling the optional gradually-narrowing end 150 of the device, to locate the device in place and/or extract the device.

In some embodiments, the optional gradually-narrowing end 150 of the device may be anchored and/or supported by a longitudinal support embodiment such as described herein.

Reference is now made to FIG. 1F, which is a simplified line drawing illustration of a device which optionally includes debris trapping such as described in above-mentioned U.S. Provisional Patent Application No. 63/398,546 which potentially benefits from anchoring according to an example embodiment.

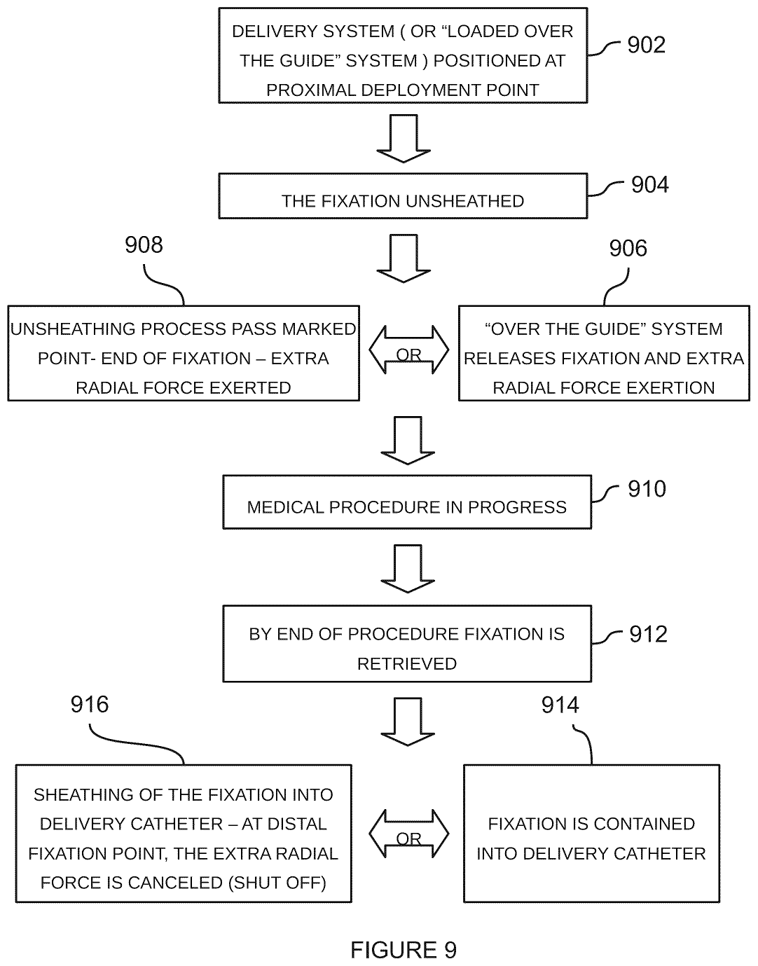

FIG. 9 shows the Vena Cava 162, and a debris trapping device 164 located in the Vena Cava 162. The Vena Cava 162 collects blood from veins to a right atrium of the heart 168. The heart pumps the blood to the lungs. If debris reaches the lungs it may be very detrimental.

In some embodiments, the debris trapping device 164 is placed in the Vena Cava 162, to block such debris. In some embodiments, the debris trapping device 164 traps debris and is eventually withdrawn from a body, together with the debris. In some embodiments, the debris trapping device 164 is optionally controlled to open and/or close debris traps by a control wire 906.

In some embodiments, the control wire 906 provides longitudinal support for the debris trapping device 904.

In some embodiments, the control wire 906 passes through a longitudinal support (not shown) which provides longitudinal support for the debris trapping device 904.

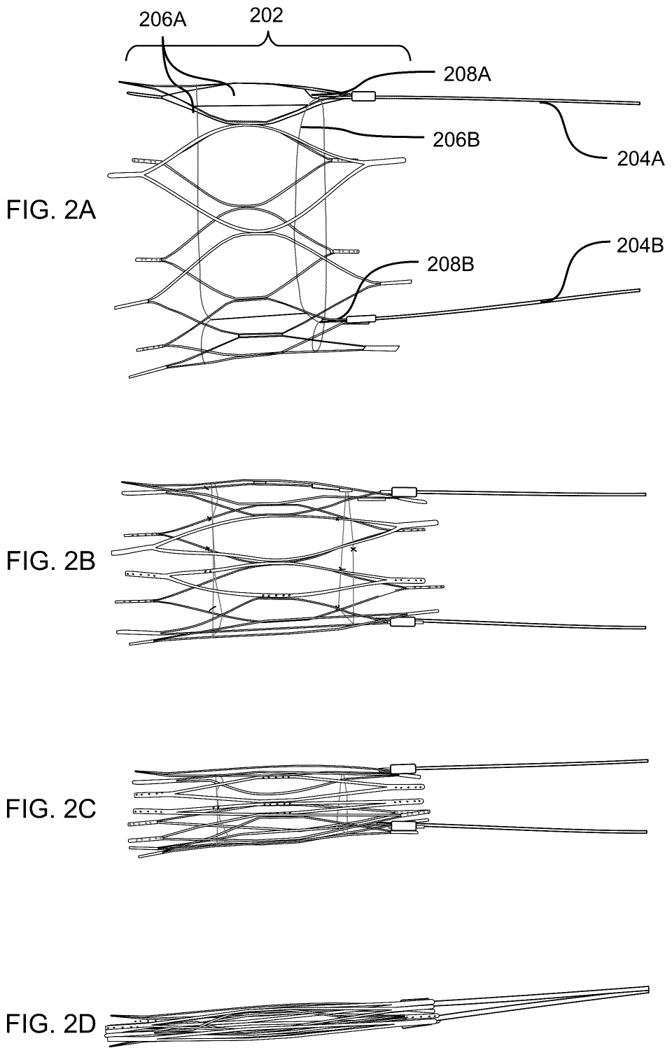

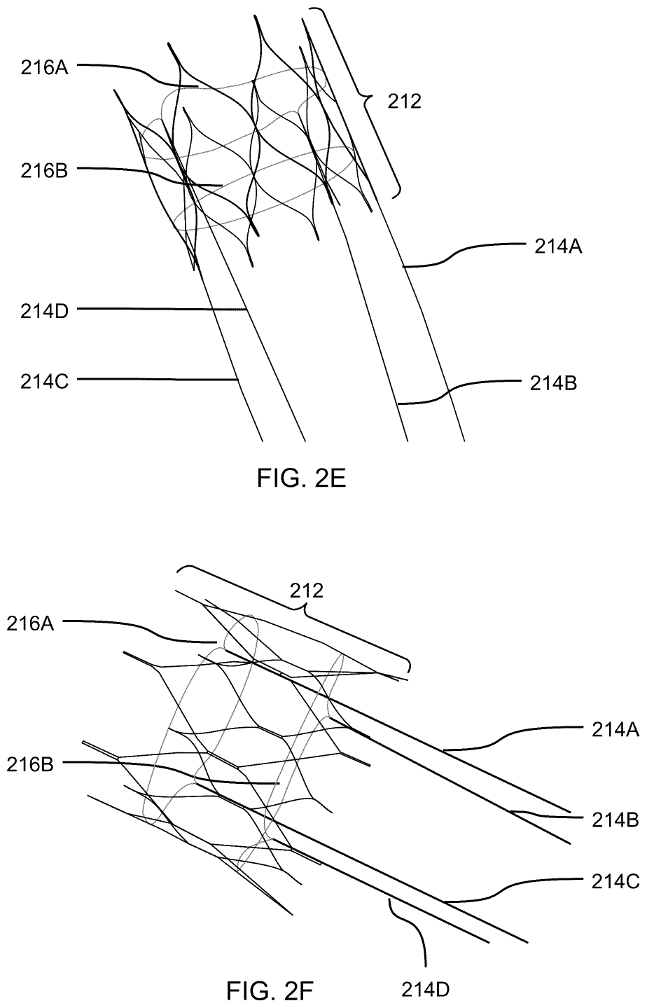

Reference is now made to FIGS. 2A-2D, which are photographs of a mechanism for anchoring according to an example embodiment.

FIGS. 2A-2D are intended to show controlled expansion of an anchoring frame mechanism, and an optional feature of longitudinal support of the anchoring frame mechanism.

FIGS. 2A-2D show an expandable anchoring frame 202, attached to two supports 204A 204B.

In some embodiments, the supports 204A 204B are hollow tubes, through which pass control wires 206A 206B. For example, in FIGS. 2A-2D, a first control wire 206A passes through a first support 204A, out of a tip 208A of the first support 204A, and extends to a control wire 206A loop around a distal side of the expandable anchoring frame 202. A second control wire 206B passes through a second support 204B, out of a tip 208B of the second support 204B, and extends to a control wire 206B loop around a distal side of the expandable anchoring frame 202.

FIGS. 2B, 2C and 2D shown the same components as FIG. 2A, however, for the sake of clarity, the Figures do not include the reference numbers, so the components are viewable more clearly.

When the first control wire 206A and/or the second control wire 206B are pulled into the first support 204A and/or the second support 204B respectively, the loops shorten and pull the anchoring frame 202 closed, as shown in the series of FIGS. 2A-2D.

Conversely, when the first control wire 206A and/or the second control wire 206B are released out of the first support 204A and/or the second support 204B respectively, the wires allow the anchoring frame 202 to expand, as may be seen when viewing the figures in an order 2D, 2C, 2B and 2A.

In some embodiments, the anchoring frame 202 includes shape memory material which exerts a force to expand, and the first control wire 206A and/or the second control wire 206B exert a force to compress the anchoring frame 202.

In some embodiments, the first support 204A and/or the second support 204B also provide longitudinal support, fixation or anchoring to the anchoring frame 202, and/or to any medical device receiving anchoring from the anchoring frame 202. The first support 204A and/or the second support 204B are optionally laterally-flexible and non-compressible longitudinally.

Reference is now made to FIGS. 2E-2F, which are photographs of a mechanism for anchoring according to an example embodiment.

FIGS. 2E-2F are intended to show an anchoring frame mechanism, and an optional feature of longitudinal support of the anchoring frame mechanism.

FIGS. 2E-2F shows an expandable anchoring frame 212, attached to four supports 214A 214D.

In some embodiments, one or more of the supports 214A 214D are hollow tubes, through which pass control wires, by way of a non-limiting example two control wires 216A 216B. For example, in FIGS. 2E-2F, a first control wire 216A passes through a first support 214A, out of the first support 214A, and extends to a control wire 216A loop around a distal side of the expandable anchoring frame 212. A second control wire 216B passes through a second support 214B, out of the second support 214B, and extends to a control wire 216B loop around a distal side of the expandable anchoring frame 212.

In some embodiments, one or more of the supports 214A-D also provide longitudinal support, fixation or anchoring to the anchoring frame 212, and/or to any medical device receiving anchoring from the anchoring frame 212. One or more of the supports 214A-D are optionally laterally-flexible and non-compressible longitudinally.

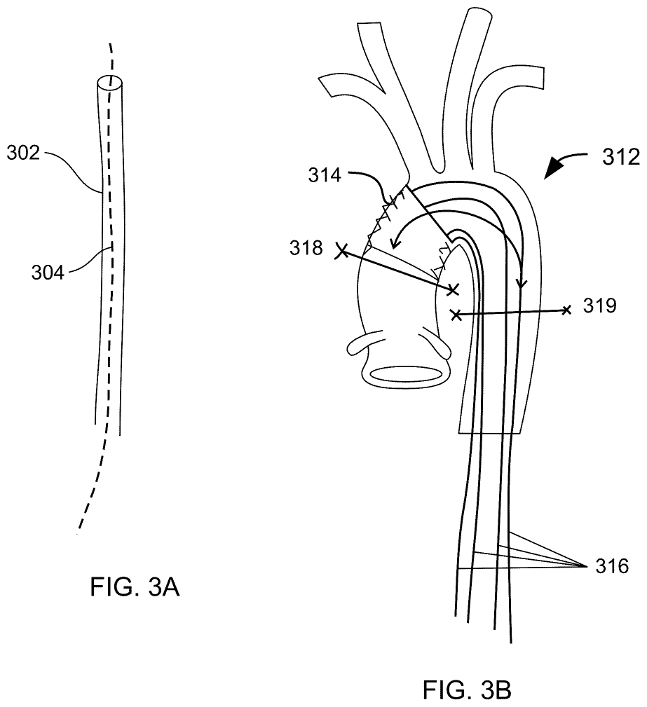

Reference is now made to FIG. 3A, which is a simplified illustration of a support and a control wire passing therethrough according to an example embodiment.

FIG. 3A show a hollow support 302 and a control wire 304 passing through the hollow support 302.

In some embodiments, the control wire 302 serves to control a medical tool and/or an anchoring device and/or anchoring frame to which the control wire 302 is connected.

In some embodiments, the support 302 is optionally laterally-flexible, in order to pass through various twists of a body lumen, and optionally longitudinally incompressible, in order to prevent longitudinal shifts of a medical device connected to the support 302.

Reference is now made to FIG. 3B, which is a simplified illustration of an anchoring frame and supports and/or control wires according to an example embodiment.

FIG. 3B shows an aorta 312, in which an anchoring frame 314 has been placed in the ascending aorta.

In some embodiments, the anchoring frame 314 is optionally controllable by one or more control wires 316 and/or longitudinal supports 316.

In some embodiments, the anchoring frame 314 is optionally provided longitudinal support by one or more longitudinal supports 316.

In some embodiments, the anchoring frame 314 may optionally be part of an aortic protection device (not shown), by way of a non-limiting example such as described in above-mentioned International Patent Application Publication Number WO 2019/064223. The aortic protection device may optionally extend along the aortic arch, by way of a non-limiting example from location 318 to location 319, protecting entrances to arteries branching off the aortic arch.

In some embodiments, activation of fixation is performed in two stage. In a first stage an anchoring frame is placed/deployed at a desired location, and a relatively lower radial force is exerted on body lumen walls. After the desirable deployment location has been verified, the anchoring frame is activated to exert extra, additional radial force.

In some embodiments, the additional force is exerted by unsheathing a delivery catheter beyond a first unsheathing which release the anchoring frame.

In some embodiments, the expanded fixation area exerts a same\equal radial pressure over the fixation area.

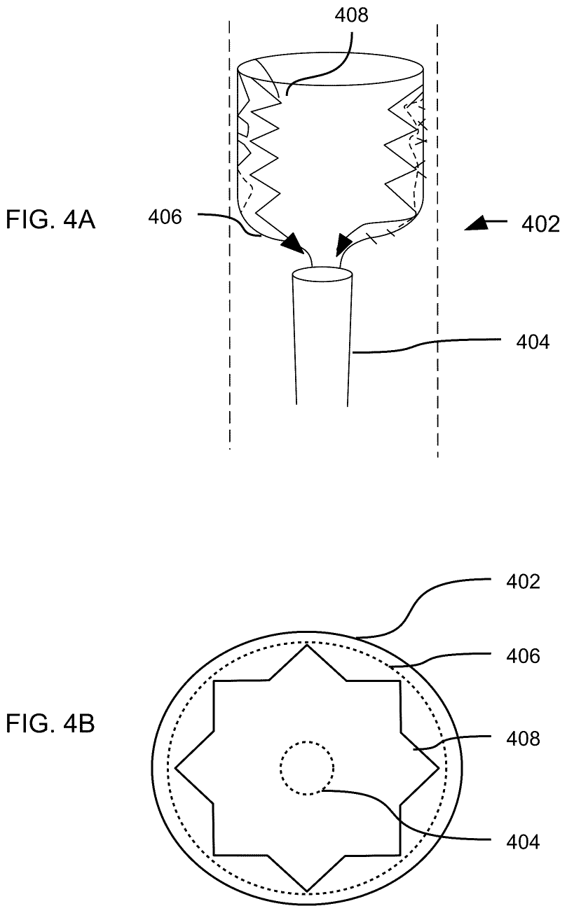

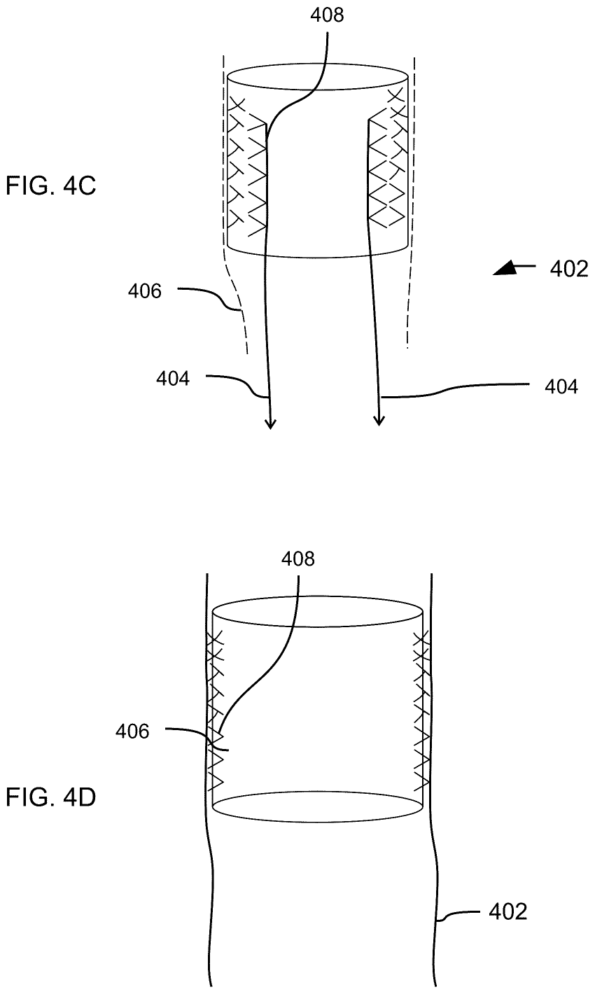

Reference is now made to FIG. 4A, which is a simplified illustration of an anchoring frame exiting a catheter according to an example embodiment.

FIG. 4A is intended to show an anchoring frame having two approximately concentric layers.

FIG. 4A shows a body lumen 402, and introducing catheter 404, and an anchoring frame having two layers-an outer layer 406 and an inner layer 408.

In some embodiments, the outer layer 406 expands and presses on walls of the body lumen 402. In some embodiments, the outer layer includes memory material configured to expand and press on walls of the body lumen 402.

In some embodiments, the outer layer 406 is configured to start expanding only under external control, for example the external control releasing the outer layer 406 to expand.

In some embodiments, the inner layer 408 expands and presses on an inner surface of the outer layer 406, increasing a pressure of the outer layer 406 on the walls of the body lumen 402.

In some embodiments, the inner layer 408 includes memory material configured to expand and press on an inner surface of the outer layer 406,

In some embodiments, the inner layer 408 is configured to start expanding only under external control, for example the external control releasing the inner layer 408 to expand.

Reference is now made to FIG. 4B, which is a simplified illustration of an anchoring frame according to an example embodiment.

FIG. 4B is intended to show a cross section of the embodiment of the example embodiment shown in FIG. 4A in the body lumen, to demonstrate the anchoring frame having two layers.

FIG. 4B shows a cross section of a body lumen 402, and an anchoring frame having two layers—an outer layer 406 and an inner layer 408.

In some embodiments, the outer layer 406 expands and presses on walls of the body lumen 402.

In some embodiments, the inner layer 408 expands and presses on an inner surface of the outer layer 406, increasing a pressure of the outer layer 406 on the walls of the body lumen 402.

In some embodiments, a cross section of the inner layer 408 is shaped as a star configured to expand and press on the outer layer 406.

In some embodiments, the outer layer 406 is optionally coated, on its outer surface with a smooth coating, optionally a polymer coating, so as not to damage the walls of the body lumen 402.

In some embodiments, the outer layer 406 is optionally coated, on both its outer surface and its inner surface with a smooth coating, optionally a polymer coating.

FIG. 4B also shows a cross section of the introductory catheter 404.

In some embodiments, the anchoring frame is configured to exert approximately equal pressure on all or most of the outer surface of the anchoring frame. Such approximately equal pressure potentially provides one or all of the following benefits:
preventing localized pressure peaks, which might damage or wound the walls of the body lumen; and
lowering pressure required, per body lumen wall area, to anchor a medical device against a specific force acting to shift a medical device anchored by the anchoring frame.

Reference is now made to FIG. 4C, which is a simplified illustration of an anchoring frame according to an example embodiment.

FIG. 4C is intended to show a side view of the embodiment of the example embodiment shown in FIG. 4A when it is released to expand in the body lumen.

FIG. 4C shows a body lumen 402, and an anchoring frame having two layers-an outer layer 406 and an inner layer 408. FIG. 4C also shows one or more control wires 404.

In some embodiments, the control wire(s) 404 are used to control expansion of the outer layer 406 and/or the inner layer 408.

In some embodiments, the control wire(s) 404 are used to control retrieving the outer layer 406 and/or the inner layer 408 into a catheter for extraction from a patient's body.

Reference is now made to FIG. 4D, which is a simplified illustration of an anchoring frame according to an example embodiment.

FIG. 4D is intended to show a side view of the embodiment of the example embodiment shown in FIG. 4A when both layers of the anchoring frame have expanded.

FIG. 4D shows a body lumen 402, and an anchoring frame having two layers-an outer layer 406 and an inner layer 408.

It is mentioned again that in some embodiments, the anchoring frame is configured to exert approximately equal pressure on all or most of the outer surface of the anchoring frame.

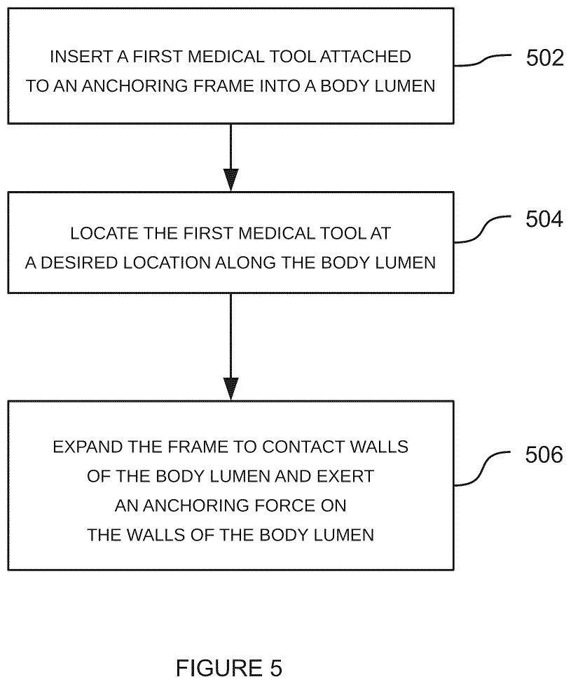

Reference is now made to FIG. 5, which is a simplified flow chart illustration of a method for anchoring a medical tool in a body lumen according to an example embodiment.

The method of FIG. 5 includes:
inserting a first medical tool attached to an anchoring frame into a body lumen (502);
locating the first medical tool at a desired location along the body lumen (504);
expanding the frame to contact walls of the body lumen and exert an anchoring force on the walls of the body lumen (506).

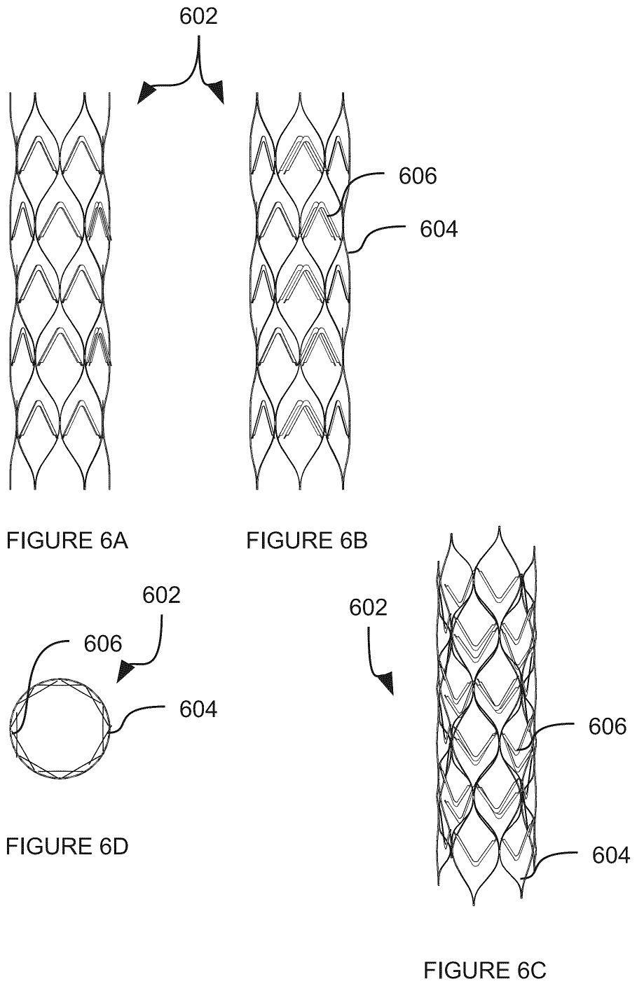

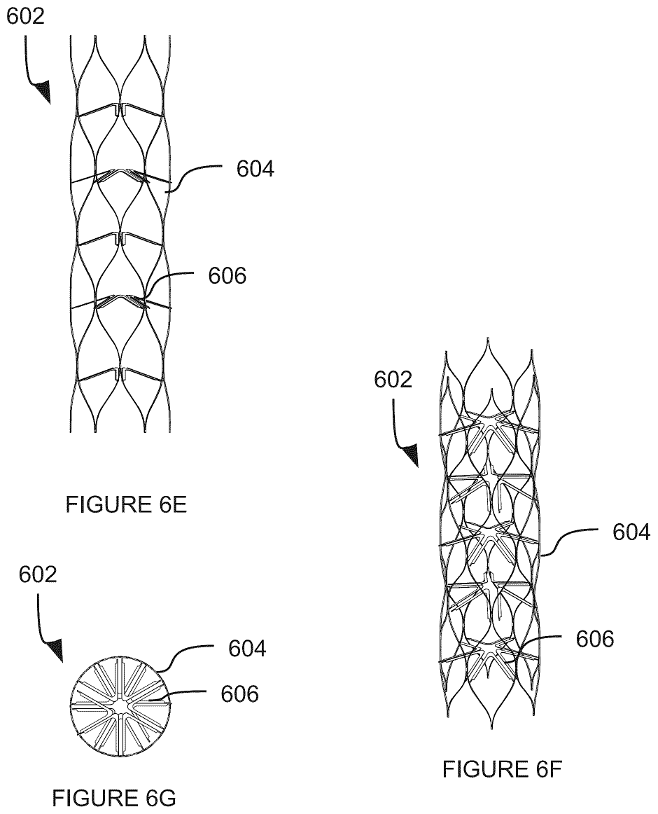

Reference is now made to FIGS. 6A-6G, which are drawings of an anchoring frame according to an example embodiment.

FIGS. 6A-6G are intended to show a frame having a two approximately concentric layers, an outer layer for pressing against walls of a body lumen, and an inner layer for exerting outward pressure on the outer layer.

FIGS. 6A-6G show an expandable frame 602, an outer layer 604, and an inner layer 606.

FIGS. 6A-6D show the frame 602 in an open state, with the inner layer 606 lying close to the outer layer 604. In this state the inner layer 606 exerts little or no pressure on the outer layer 604.

FIGS. 6A and 6B are side views, FIG. 6C is an isometric view, and FIG. 6D is a cross section view along the frame 602.

FIGS. 6E-6G show the frame 602 in an activated or closed state, with the inner layer 606 pushing radially outwards on the outer layer 604. In this state the inner layer 606 exerts more pressure on the outer layer 604.

FIG. 6E is a side view, FIG. 6F is an isometric view, and FIG. 6G is a cross section view along the frame 602.

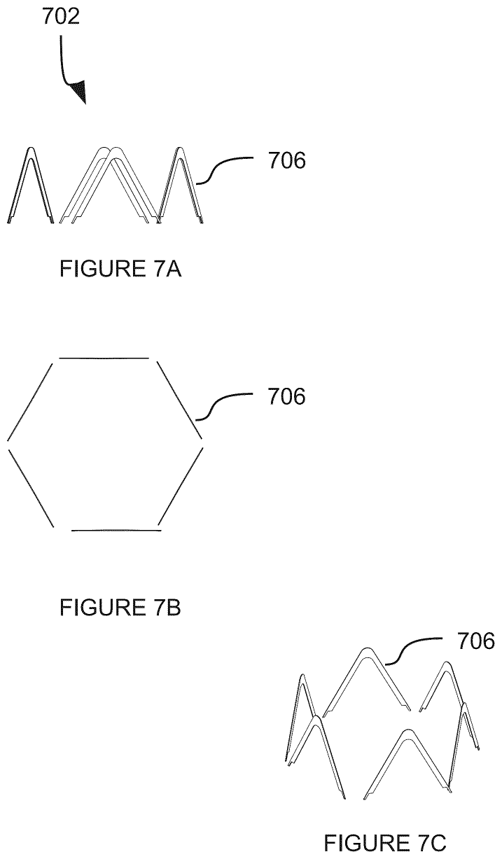

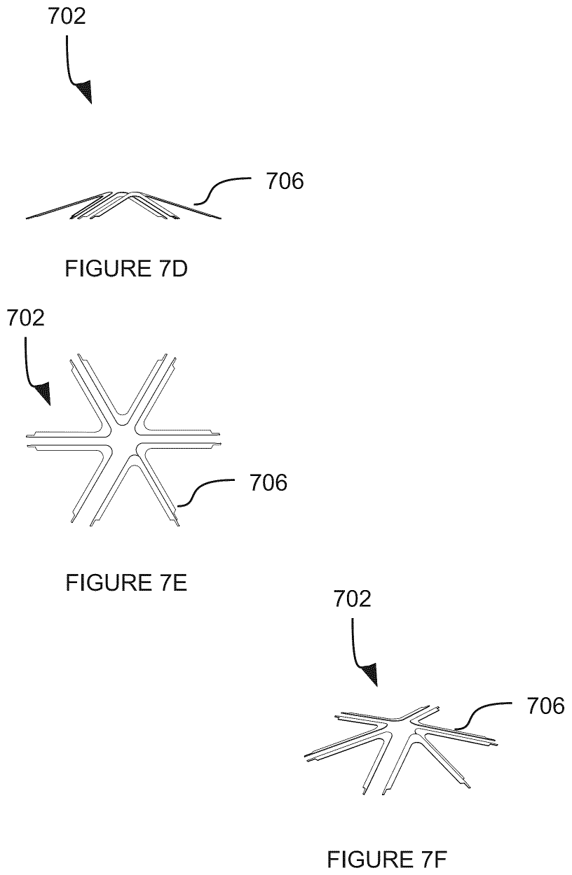

Reference is now made to FIGS. 7A-7F, which are drawings of an inner layer of an anchoring frame according to an example embodiment.

FIGS. 7A-7F are intended to show an inner layer of an expandable frame which has two layers, an outer layer for pressing against walls of a body lumen, and an inner layer for exerting outward pressure on the outer layer.

FIGS. 7A-7F an expandable frame 702, and components of an inner layer 706.

FIGS. 7A-7C the inner layer 606 in an open state, exerting little or no pressure on an outer layer (not shown).

FIGS. 7A is a side view, FIG. 7B is a cross section view along the frame 702, and FIG. 7C is an isometric view.

FIGS. 7D-7F show the frame 702 in an activated or closed state, with the components of the inner layer 706 arranged to push radially outwards on an outer layer (not shown). In this state the components of the inner layer 706 exerts more pressure on the outer layer.

FIG. 7D is a side view, FIG. 7E is a cross section view along the frame 602, and FIG. 7F is an isometric view.

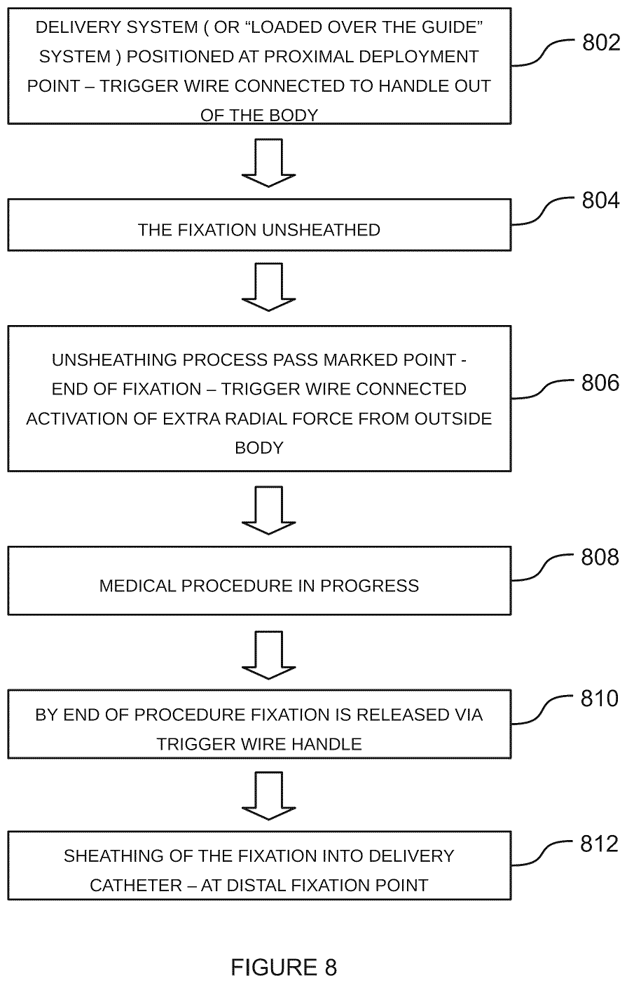

Reference is now made to FIG. 8, which is a simplified flow chart illustration of a method of fixation including controlling an addition of additional, extra, radial force according to an example embodiment.

The method of FIG. 8 includes:
positioning a delivery system (or a “loaded over a guide” system) at a proximal deployment point—with a trigger wire connected to a handle extending out of a patient's body (802); unsheathing the fixation (804);
the unsheathing process passing a marked point—end of fixation—trigger wire connected. Causes activation of extra radial force from outside the patient's body (806);
performing a medical procedure (808);
releasing fixation via trigger wire handle when medical procedure has ended (810); and
sheathing the fixation into a delivery catheter as a distal fixation point (812).

It is noted that sheathing and/or collapsing the fixation circumferentially before retrieving the fixation out of a patient's body potentially provides some or all of the following benefits:
refraining from wounding and/or injuring the patient by dragging the fixation along a body lumen; and
refraining from releasing debris by dragging the fixation along walls of a body lumen.

Reference is now made to FIG. 9, which is a simplified flow chart illustration of a method of fixation including self-expansion and controlling an addition of additional, extra, radial force according to an example embodiment.

The method of FIG. 9 includes:
positioning a delivery system (or a “loaded over a guide” system) positioned at a proximal deployment point (902);
unsheathing the fixation (904);
EITHER the “loaded over a guide” system release fixation and extra radial force is exerted (906) OR the unsheathing process passing a marked point-end of fixation-causes activation of extra radial force (908);
performing a medical procedure (910);
retrieving the fixation at the end of the medical procedure (912) by EITHER containing the fixation into a delivery catheter (914) OR sheathing the fixation into a delivery catheter at a distal fixation point, and the extra radial force is cancelled, that is, shut off (916).

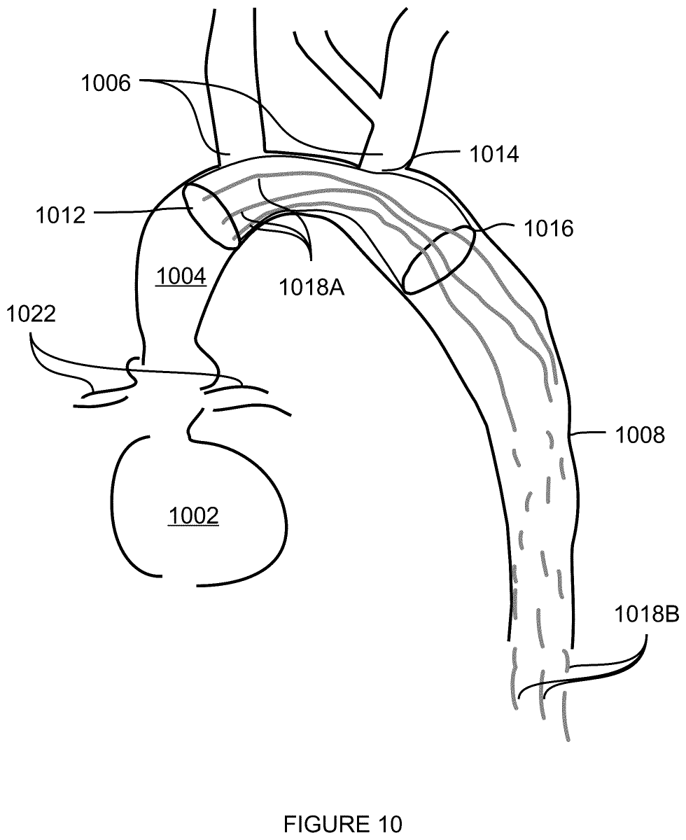

Reference is now made to FIG. 10, which is a simplified line drawing illustration of a debris protection device which potentially benefits from anchoring according to an example embodiment.

FIG. 10 shows a heart 1002, coronary arteries 1022, ascending aorta 1004, arteries 1006 leading to the brain, and descending aorta 1008. FIG. 10 also shows a debris protection device 1014, having a distal end 1012 and a proximal end 1016.

In some embodiments the debris protection device 1014 includes control wires 1018B, and the control wires pass through hollow tubes 1018A. The hollow tubes 1018A are optionally axially inflexible and can bend laterally, and optionally act as elongate support keeping the debris protection device 1014.

In some embodiments the hollow tubes 1018A extend all the way outside a patient's body, and can be controlled and maneuvered forward and backward, to move the debris protection device 1014 and/or held in place to support the debris protection device 1014 at a desired location.

In some embodiments, the hollow tubes 1018A are connected to the filter at or near a distal end of the tube.

In some embodiments, a number of the hollow tubes 1018A may be in a range including 1, 2, 3, 4, 5, 6, and on up to 10, 12, 16 and even 20.

In some embodiments, a material of which the hollow tubes 1018A are composed of a metal such as Nitinol, and/or a polymer, and/or other materials approved for us within a body, and providing axial support.

It is expected that during the life of a patent maturing from this application many relevant devices which can use fixation as described herein, such as debris-protection devices and debris capture devices will be developed and the scope of the terms medical devices which can use fixation, debris-protection devices and debris capture devices are intended to include all such new technologies a priori.

As used herein with reference to quantity or value, the terms “about” and approximately mean “within ±25% of”.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A device for anchoring a medical tool in a large body lumen, the device comprising a support configured to axially support said medical tool in a body lumen against longitudinal movement along the body lumen, wherein:
the support is axially inflexible;
the support can bend laterally; and
the support is configured to connect to said medical tool to anchor the medical tool in the body lumen; and
wherein said support comprises a hollow tube and said hollow tube contains a control wire for controlling said medical tool.

2. (canceled)

3. The device according to claim 1, wherein the large body lumen is an aorta and the device is shaped and sized to extend across an ascending aorta.

4. (canceled)

5. The device according to claim 1, wherein the large body lumen is a Vena Cava and the device is shaped and sized to extend across the Vena Cava.

6. The device according to claim 1, wherein the device comprises a plurality of supports.

7-8. (Cancelled)

9. The device according to claim 1, wherein the medical tool comprises a blood filtration mesh.

10. The device according to claim 1, wherein the medical tool comprises a device for trapping debris flowing along the body lumen.

11. The device according to claim 1, wherein the medical tool comprises a device for trapping debris located in a vein.

12. The device according to claim 1, wherein the medical tool comprises a device for trapping debris located in a Vena Cava.

13. A system comprising a filter and a device for anchoring a medical tool in a large body lumen; said device comprising a support configured to axially support a medical tool in a body lumen against longitudinal movement along the body lumen, wherein:
the support is axially inflexible;
the support can bend laterally; and
the support is configured to connect to a medical tool to anchor the medical tool in the body lumen;
wherein said support comprises a hollow tube and said hollow tube contains a control wire for controlling said medical tool.

14. The system according to claim 13, wherein the support is connected to the filter at or near a distal end of the support.

15. A method for anchoring a medical tool in a body lumen, the method comprising pushing a first medical tool supported by a support into a body lumen,
wherein the support is longitudinally inflexible and laterally flexible, preventing longitudinal movement of the first medical tool along the body lumen.

16. The method according to claim 15, wherein the first medical tool comprises a filter.

17. The method according to claim 15, wherein the support is attached to the first medical tool.

18. The method according to claim 17, wherein the hollow tube contains a control wire for controlling a second medical tool.

19. The method according to claim 15, wherein the medical tool comprises a device for blocking debris from entering coronary arteries.

20. The method according to claim 15, wherein the medical tool comprises a device for blocking debris from entering arteries leading to a brain.

21. The method according to claim 15, wherein the medical tool comprises a device for trapping debris located in a vein.

22. The method according to claim 15, wherein the medical tool comprises a device for trapping debris located in a Vena Cava.

23-50. (canceled)
